(19) United States
(12) Patent Application Publication
(10) Pub. No.: US 2018/0028052 A1
(43) Pub. Date: Feb. 1, 2018

(54) ENDOSCOPE SYSTEM

(71) Applicant: OLYMPUS CORPORATION, Tokyo (JP)

(72) Inventor: Hiroyuki KOJO, Hachioji-shi (JP)

(73) Assignee: OLYMPUS CORPORATION, Tokyo (JP)

(21) Appl. No.: 15/220,495

(22) Filed: Jul. 27, 2016

(51) Int. Cl. A61B 1/00; A61B 5/06; A61B 34/00; A61B 1/05; A61B 1/005

(52) U.S. Cl. CPC A61B 1/00154; A61B 1/05; A61B 1/0052; A61B 34/76; A61B 1/00045; A61B 5/065; A61B 1/00055

(57) ABSTRACT

An endoscope system includes, a holding unit; a guide pipe extending from the holding unit, an endoscope including a distal end portion capable of acquiring an image, the endoscope being inserted through an inside of the guide pipe and bendable in at least one plane, a bending operation unit rotatably provided on the holding unit and capable of adjusting a bending angle of the endoscope, and a pointer provided on the bending operation unit and indicating an extending direction of the distal end portion in the plane.

### FIELD OF THE INVENTION

Embodiments described herein relate generally to an endoscope system capable of visually recognizing the inside of a cavity of a subject.

### DESCRIPTION OF THE RELATED ART

For example, Japanese Patent Application Publication No. 5-15484 discloses an endoscope capable of quantitatively recognizing an angle of a distal end portion of an inserting tube in the body cavity of the subject from outside of the subject.

### BRIEF SUMMARY OF THE INVENTION

An endoscope comprises, a holding unit, a guide pipe extending from the holding unit, an endoscope including a distal end portion capable of acquiring an image, the endoscope being inserted through an inside of the guide pipe and bendable in at least one plane, a bending operation unit rotatably provided on the holding unit and capable of adjusting a bending angle of the endoscope; and a pointer provided on the bending operation unit and indicating an extending direction of the distal end portion in the plane.

Advantages of the invention will be set forth in the description which follows, and in part will be obvious from the description, or may be learned by practice of the invention. Advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram illustrating a whole structure of an endoscope system according to an embodiment, and illustrating an inside of a guide pipe by cutting the guide pipe;

FIG. 2 is a schematic diagram illustrating an inside of a holding unit of the endoscope system illustrated in FIG. 1 that is cut along line F2-F2;

FIG. 3 is a schematic diagram schematically illustrating a distal end portion and therearound of an endoscope inserting unit of the endoscope system illustrated in FIG. 1;

FIG. 4 is a schematic diagram schematically illustrating that an extending direction of the distal end portion agrees with an indicating direction of a pointer, when a dial portion of a bending operation unit is rotated to bend a flexible tube of the endoscope inserting unit, in the endoscope system illustrated in FIG. 1;

FIG. 5 is a schematic diagram illustrating a whole structure of an endoscope system according to a first modification, and illustrating an inside of a guide pipe by cutting the guide pipe;

FIG. 6 is a schematic diagram illustrating a whole structure of an endoscope system according to a second modification, and illustrating an inside of a guide pipe by cutting the guide pipe; and

FIG. 7 is a schematic diagram schematically illustrating that an extending direction of a distal end portion agrees with an indicating direction of a second pointer of a second dial portion, in a state where the distal end portion of an endoscope inserting unit is caused to widely project from the guide pipe, in the endoscope system illustrated in FIG. 6.

### DETAILED DESCRIPTION OF THE INVENTION

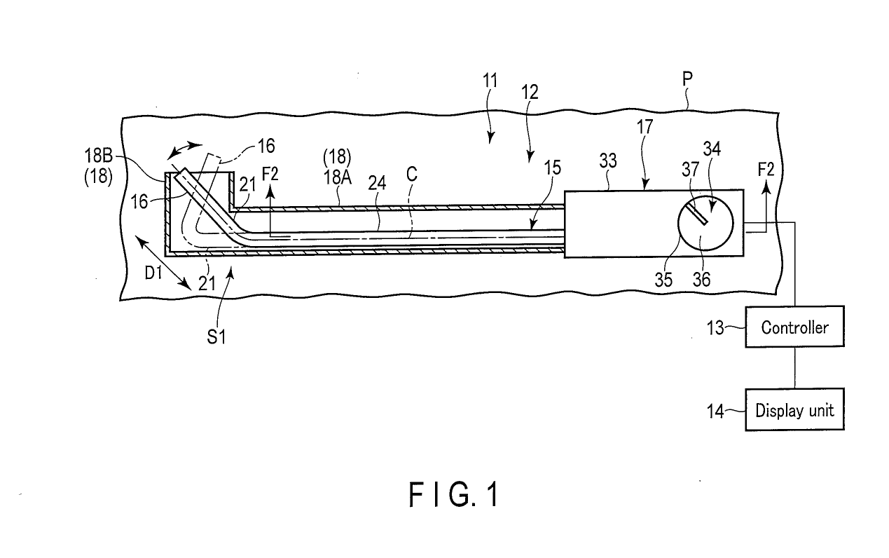

An embodiment will be explained hereinafter with reference to FIG. 1 to FIG. 4.

As illustrated in FIG. 1, an endoscope system 11 according to the present embodiment includes an inserting device 12 that is inserted to be used into a cavity (such as a paranasal sinus) of the subject, a controller 13 that is connected with the inserting device 12, and a display unit 14 that is connected with the controller. The inserting device 12 is provided separately from the display unit 14 and the controller 13. The display unit 14 is formed of an ordinary liquid crystal monitor, and capable of displaying an image acquired by a distal end portion 16 of an endoscope 15 as an image.

As illustrated in FIG. 1 and FIG. 3, the inserting device 12 includes a holding unit 17 serving as an outer shell of the device, a cylindrical guide pipe 18 projecting from the holding unit 17, an endoscope inserting unit 21 inserted through the inside of the guide pipe 18 and the holding unit 17, an endoscope imaging unit 22 (imaging unit) provided inside the holding unit 17, and a bending operation unit 34 provided on the holding unit 17. In the present embodiment, although the endoscope 15 is separated into two units serving as the endoscope inserting unit 21 and the endoscope imaging unit 22, the endoscope 15 may be formed by uniting these units.

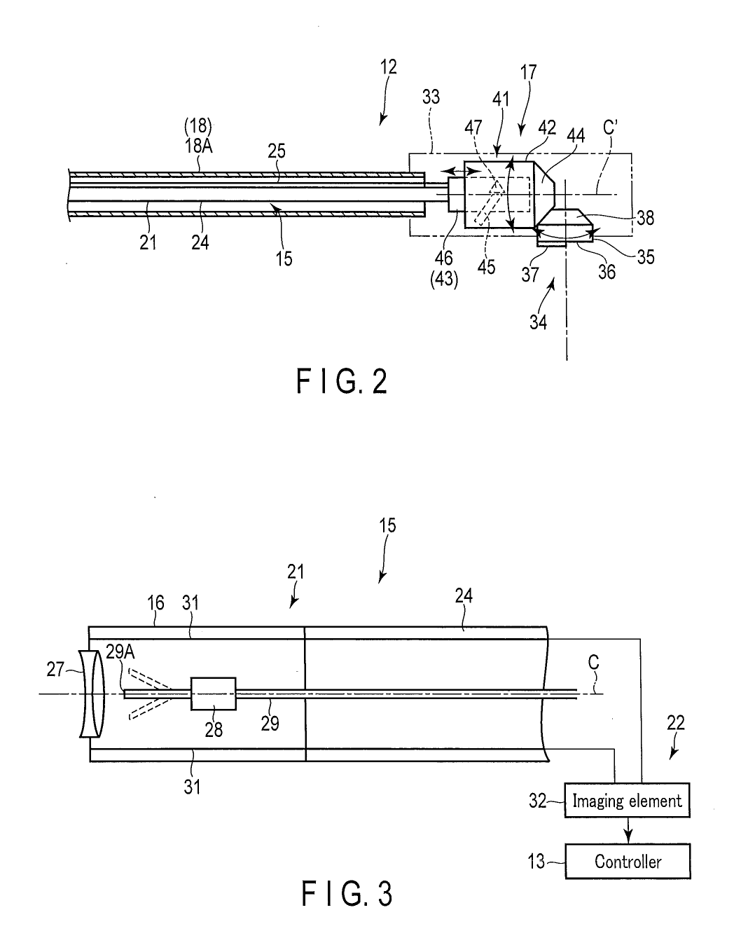

The endoscope 15 is formed of a scanning endoscope. The whole endoscope 15 (endoscope inserting unit 21) is configured to have flexibility. With such structure, the endoscope inserting unit 21 inserted into the guide pipe 18 can be bent to follow the shape of the guide pipe 18. As illustrated in FIG. 2 and FIG. 3, the endoscope inserting unit 21 includes a central axis C. The endoscope inserting unit 21 is movable along the central axis C, and can be caused to project from the guide pipe 18.

As illustrated in FIG. 1 to FIG. 3, the endoscope inserting unit 21 includes a distal end portion 16 (distal end forming portion) positioned on the distal end side thereof in the direction of the central axis C, a flexible tube 24 provided on the proximal end side in the direction of the central axis C from the distal end portion 16, a wire 25 (pull wire) connected to a distal end side of a sheath, a cylindrical sheath covering the distal end portion 16, the flexible tube 24, and the wire 25, an illumination window 27, a rotating unit 28, an illumination fiber 29, and a plurality of light-receiving fibers 31. In the present embodiment, illustration of the sheath is omitted. The sheath has a structure capable of being bent as illustrated in FIG. 1, together with the endoscope inserting unit 21 (flexible tube 24) that is held inside thereof. The sheath is bent by pulling the wire 25 with a holding portion 43 provided on the bending operation unit 34 side. By bending the sheath, the endoscope inserting unit 21 (flexible tube 24) disposed inside thereof is bent.

The illumination fiber 29 is optically connected with a light source provided adjacent to the controller 13. The light-receiving fibers 31 are optically connected with an imaging element 32. Distal ends of the light-receiving fibers 31 are exposed to the outside at the distal end portion 16. With such structure, the endoscope 15 is enabled to acquire an image at the distal end portion 16 via the light-receiving fibers 31.

The wire 25 is provided inside the sheath and outside the flexible tube 24, and extends from the holding unit 17 to the distal end portion 16. The wire 25 is pulled to adjust the bending angle of the endoscope 15 (endoscope inserting unit 21). The wire 25 is an example of a linear member. The distal end of the wire 25 is fixed to the distal end portion 16 (distal end forming portion). The proximal end of the wire 25 is fixed to the holding portion 43 described later. By performing an operation to move the wire 25 forward or backward in the holding unit 17, the endoscope inserting unit 21 can be bent in a portion in the flexible tube 24 close to the distal end portion 16.

Pulling with the wire 25 corresponds to a bend in the right direction (clockwise direction) in FIG. 1, for example. A bend in the left direction (counterclockwise direction) in FIG. 1 is achieved by a restoring force of the endoscope inserting unit 21 returning from a bent state pulled by the wire 25 to its original straight state. Two wires may be provided to bend the endoscope inserting unit 21, as a matter of course. The two wires include a first wire corresponding to a rightward bend in FIG. 1, and a second wire corresponding to a leftward bend in FIG. 1.

As illustrated in FIG. 2, the endoscope imaging unit 22 includes the imaging element 32 formed of a CCD or a CMOS. The endoscope imaging unit 22 is capable of acquiring an image acquired at the distal end portion 16 of the endoscope inserting unit 21. More specifically, the imaging element 32 converts light from the light-receiving fibers 31 into an electrical signal, and transmits the signal to the controller 13.

The rotating unit 28 is electrically connected with the controller 13. The rotating unit 28 is formed of a motor or the like, and is rocked in a spiral manner, for example, by the controller 13. With this structure, a distal end 29A of the illumination fiber 29 is rocked in a spiral manner in accordance with the operation of the rotating unit 28. Accordingly, the surface of the subject to be imaged is scanned in a spiral manner with the illumination light from the illumination fiber 29, through the distal end 29A of the illumination fiber 29 and the illumination window 27. The light-receiving fibers 31 receive returning light from the subject, and guide the light to the imaging element 32. The imaging element 32 transmits the light received by the light-receiving fibers 31 as an electrical signal to the controller 13. The controller 13 converts the electrical signal into an image, performs proper image processing on the image, and displays the image on the display unit 14.

As illustrated in FIG. 1, the guide pipe 18 has an “L” shape as a whole, and has a cylindrical shape that is bent in an elbow shape in a middle portion. The endoscope inserting unit 21 is inserted through the inside of the guide pipe 18. An internal wall of the guide pipe 18 is capable of guiding the endoscope inserting unit 21 that is moved forward and backward along the central axis C.

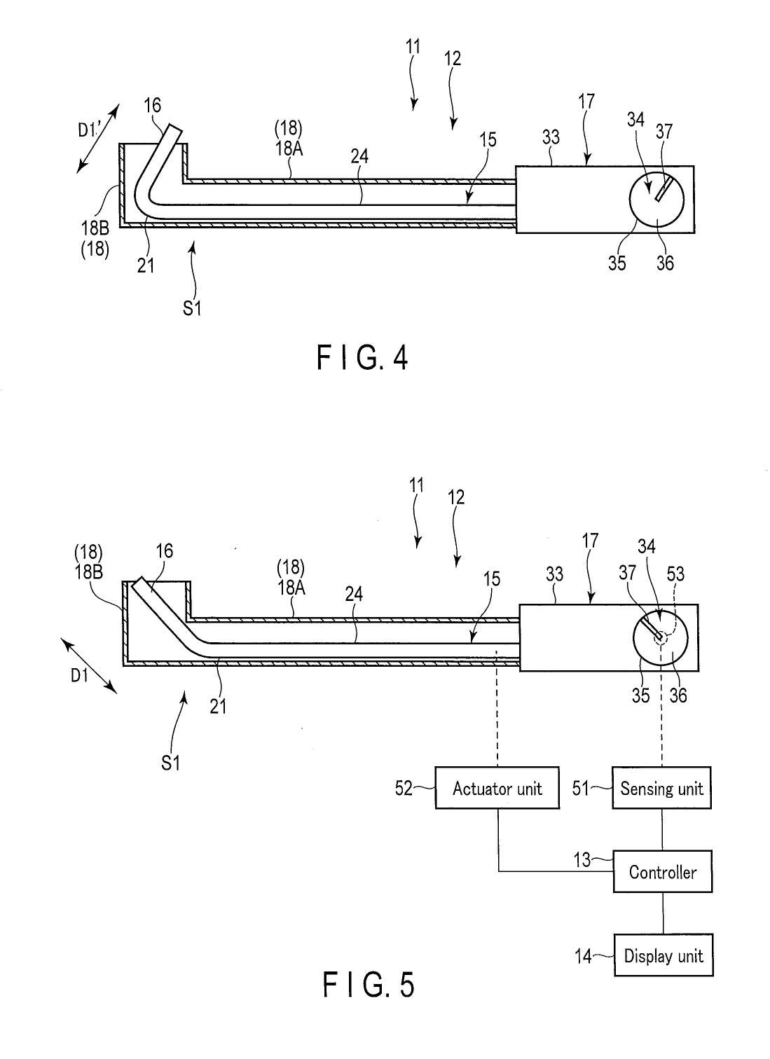

The guide pipe 18 is rotatable with respect to the holding unit 17, for example. The guide pipe 18 includes a first portion 18A that is connected with the holding unit 17, and a second portion 18B that extends in a direction crossing the direction in which the first portion 18A extends. The internal diameter of the second portion 18B is formed larger than the internal diameter of the first portion 18A, and the endoscope inserting unit 21 can be operated to be bent inside the second portion 18B (see FIG. 1 and FIG. 4). In the operation, the endoscope inserting unit 21 is rotatable inside the second portion 18B, while finely adjusting the position thereof back and forth in the direction of the central axis C. In other words, the endoscope inserting unit 21 is bendable in at least one plane P including the central axis C. The endoscope inserting unit 21 may be bendable in another plane different from one plane P, as well as in the plane P.

The holding unit 17 has a cylindrical shape, and forms a portion that is held by the user's hand. The holding unit 17 includes a housing 33, and the bending operation unit 34 capable of adjusting the bending angle of the endoscope 15.

As illustrated in FIG. 1 and FIG. 2, the bending operation unit 34 includes a dial portion 35 that is rotatable with respect to the housing 33, and a converting portion 41 that converts rotation of the dial portion 35 into forward and backward movement of the wire 25. The dial portion 35 includes a dial surface 36 that is formed flat, a pointer 37 (pointer projecting portion) that is provided to project from the dial surface 36, and a first rotation transmitting portion 38 (such as a gear (gear wheel)) provided on a side opposite to the dial surface 36. The pointer 37 is formed as a straight-line projecting portion to cross the dial surface 36. The direction in which the pointer 37 extends can indicate an extending direction D1 of the distal end portion 16 of the endoscope 15 (endoscope inserting unit 21). The concept of the bending operation unit 34 may include the controller 13. The dial portion 35 may have any structure other than the dial, as long as it has the structure rotatable with respect to the bending operation unit 34. For example, the dial portion 35 may have a structure of a rotary member (knob) that is rotatable with respect to the bending operation unit 34, as a matter of course.

The pointer 37 projects from a surrounding portion of the dial surface 36. This structure enables the user to recognize the indicating direction of the pointer 37 by feeling at the time when the user touches the pointer 37 by the finger, that is, tactually, not visually. The shape of the pointer 37 is an example, and may be another shape, as long as the user can tactually recognize the indicating direction. Specifically, for example, instead of the straight-line shape, the pointer 37 may have a curved shape that enables the user to easily operate the pointer 37, or an intermittent straight-line shape in which fine projecting portions are successively aligned, or the flat dial surface 36 may be depressed to form the pointer 37. When the bending direction of the endoscope inserting unit 21 is not two directions in one plane P but four directions in one plane P and another plane different from the plane P, the bending operation unit 34 may be achieved with a structure such as a joystick.

The converting portion 41 includes a substantially cylindrical cam cylinder 42 contained in the housing 33, and the substantially cylindrical holding portion 43 (wire holding portion) provided inside the cam cylinder 42. The cam cylinder 42 is provided with a second rotation transmitting portion 44 (such as a gear (gear wheel)) engaged with the first rotation transmitting portion 38. As an example, the first rotation transmitting portion 38 and the second rotation transmitting portion 44 are formed as a wheel train of a bevel gear, but may be formed of other common motive power transmitting units (wheel train).

The internal surface of the cam cylinder 42 is provided with one or more cam grooves 45 having a spiral shape around a central axis C′ of the cam cylinder 42. The proximal end of the wire 25 is held (fixed) on the holding portion 43. The holding portion 43 includes a holding portion main body 46 having a cylindrical shape, and a pin 47 that projects from the holding portion main body 46 outward in the radial direction of the holding portion main body 46. A distal end portion of the pin 47 is received in the cam groove 45, and is slidable in the cam groove 45. Specifically, when the dial portion 35 is rotated by the user, the cam cylinder 42 to which motive power is transmitted is rotated via the first rotation transmitting unit 38 and the second rotation transmitting unit 44. In this manner, the holding portion 43 is moved forward or backward by the cam mechanism via the cam groove 45 and the pin 47. In this manner, the wire 25 is pulled or returned to its original position, and the flexible tube 24 is bent or returned to its original position (initial state) in the vicinity of the distal end portion 16.

In the present embodiment, the inclination of the cam groove 45, and the gear ratio of the first rotation transmitting portion 38 and the second rotation transmitting portion 44 are set such that the extending direction D1 of the distal end portion 16 of the endoscope inserting unit 21 (endoscope 15) agrees with the indicating direction of the pointer 37 of the bending operation unit 34.

The holding unit 17 may be further provided with a moving mechanism to move backward and forward the endoscope inserting unit 21 with respect to the guide pipe 18 in the direction of the central axis C. Specifically, in a medical examination, the doctor who is the user can change the position of the endoscope inserting unit 21 using the moving mechanism or the like, or change the bending angle of the endoscope inserting unit 21 as described above, in the state where the endoscope inserting unit 21 is inserted into the cavity of the subject, to acquire a desired image in the cavity.

The controller 13 illustrated in FIG. 1 is formed of, for example, an ordinary computer, and software installed in the computer and performing various types of control on the inserting device 12. The controller 13 is capable of performing the following controls, for example, on constituent elements of the inserting device 12. The controller 13 is capable of controlling the rotating unit 28 that rocks the illumination fiber 29, to adjust the rotational frequency thereof or the like. The controller 13 is capable of controlling the light source, to adjust the light quantity to be supplied to the illumination fiber 29. The controller 13 is capable of converting an electrical signal corresponding to an image acquired by the imaging element 32 of the inserting device 12 into an image, and displaying the image on the display unit 14.

The following explains the operation of the endoscope system 11 according to the present embodiment, with reference to FIG. 1 and FIG. 4.

In a medical examination, the doctor who is the user can insert the endoscope inserting unit 21 into the cavity (lumen) of the medical examinee (subject), to observe the inside of the cavity. As illustrated in FIG. 1, the extending direction D1 of the distal end portion 16 of the endoscope inserting unit 21 in the initial state agrees with the indicating direction of the pointer 37. In this state, the user can bend the flexible tube 24 of the endoscope inserting unit 21 using the bending operation unit 34, to change the extending direction of the distal end portion 16 to a desired direction in one plane P.

When the dial portion 35 is rotated, for example, clockwise from the initial state illustrated in FIG. 1, the endoscope inserting unit 21 is bent rightward together with the sheath, by the operation of the cam mechanism of the converting portion 41, as illustrated in FIG. 4 (or as illustrated with a broken line in FIG. 1). In this state, even after the endoscope inserting unit 21 is bent as illustrated in FIG. 4, an extending direction D1′ of the distal end portion 16 of the endoscope inserting unit 21 agrees with the indicating direction of the pointer 37 of the bending operation unit 34. The same is also applicable to the case where the dial portion 35 is rotated, for example, counterclockwise from the initial state.

With this structure, even in the state where the endoscope inserting unit 21 is inserted into the cavity of the medical examinee, the doctor who is the user is enabled to recognize the extending direction D1 of the distal end portion 16, by checking the indicating direction of the pointer 37. In addition, because the user is enabled to recognize the indicating direction of the pointer 37 by touch (that is, tactile sensation) at the time when the pointer 37 contacts the user's hand, the user is enabled to recognize the direction observed with the distal end portion 16 without taking the his or her eyes off the display unit 14.

When the guide pipe 18 is rotated with respect to the holding unit 17, the extending direction D1 of the distal end portion 16 does not strictly agree with the indicating direction of the pointer 37. However, even in such a case, the bending amount of the flexible tube 24 agrees with the rotation angle of the pointer 37 from the initial state, and the rotation angle of the pointer 37 can indicate the bending amount of the flexible tube 24. In this case, the user is enabled to recognize the bending amount of the flexible tube 24, based on by what amount the pointer 37 is rotated from the initial state illustrated in FIG. 1.

The following can be said according to the embodiment. Specifically, the endoscope system 11 includes the holding unit 17, the guide pipe 18 extending from the holding unit 17, the endoscope 15 including the distal end portion 16 that is capable of acquiring an image, inserted through inside of the guide pipe 18 and bendable in at least one plane P, the bending operation unit 34 rotatably provided on the holding unit 17 and capable of adjusting a bending angle of the endoscope 15, and the pointer 37 provided on the bending operation unit 34 and indicating the extending direction D1 of the distal end portion 16 in the plane P.

With this structure, because the extending direction D1 of the distal end portion 16 can be recognized with the pointer 37, the user is enabled to intuitively recognize the direction observed with the distal end portion 16, even in the state where the endoscope inserting unit 21 is inserted into the cavity of the subject. This structure prevents the user from confusing the orientation during observation of the inside of the cavity of the subject, and improves the user's convenience. In addition, even when the user performs an operation of twisting the holding unit 17 around the central axis thereof, the bending operation unit 34 and the pointer 37 move together with the holding unit 17 in one-to-one correspondence. This structure enables the user to intuitively recognize the direction in which the endoscope 15 faces.

In the present embodiment, the indicating direction of the pointer 37 can be tactually recognized. This structure enables the user to recognize the indicating direction of the pointer 37 without visually checking the pointer 37, and improves the user's convenience.

The endoscope 15 includes a linear member to adjust the bend thereof, the bending operation unit 34 includes the dial portion 35 that is rotatably provided on the holding unit 17 and provided with the pointer 37, and the converting portion 41 that converts rotation of the dial portion 35 into forward or backward movement of the linear member. This structure enables agreement between the indicating direction of the pointer 37 and the extending direction D1 of the distal end portion 16, with the simple structure using the dial portion 35 and the converting portion 41. This structure reduces the manufacturing cost of the endoscope system 11, and reduces the possibility of causing a malfunction in which the indicating direction of the pointer 37 does not agree with the extending direction D1 of the distal end portion 16.

The endoscope system 11 includes the display unit 14 capable of displaying the image acquired by the distal end portion 16. This structure enables the user to intuitively recognize the extending direction of the distal end portion 16 with the pointer 37, in the state of checking the image displayed on the display unit 14. This structure causes the user's next operation of directing the endoscope 15 in the desired direction to be more intuitive, while the user is viewing the display unit 14, and shortens the diagnostic time. These structures improve the user's convenience, and reduces the burden on the medical examinee (subject).

The following explains a first modification of the present embodiment, with reference to FIG. 5. The first modification explained hereinafter mainly illustrates parts that are different from the present embodiment, and explanation of parts common to the embodiment and the modification is omitted. In the embodiment, the extending direction of the distal end portion 16 of the endoscope inserting unit 21 is mechanically caused to agree with the indicating direction of the pointer 37. In the present modification, the extending direction D1 of the distal end portion 16 of the endoscope inserting unit 21 is caused to agree with the indicating direction of the pointer 37 by electrical control.

The holding unit 17 includes the housing 33, and the bending operation unit 34 capable of adjusting the bending angle of the endoscope 15.

The bending operation unit 34 includes the dial portion 35 rotatable with respect to the housing 33, a sensing unit 51 sensing the rotation amount of the dial portion 35, an actuator unit 52 to move the wire 25 forward and backward to bend the endoscope inserting unit 21 in one plane P, and the controller 13. The dial portion 35 includes the dial surface 36 that is formed flat, a pointer 37 (pointer projecting portion) provided to project from the dial surface 36, and a rotation axis 53 provided on a side opposite to the dial surface 36. The sensing unit 51 is formed of, for example, a potentiometer, and is capable of reading the rotation amount (rotation angle) of the rotation axis 53.

The actuator unit 52 is formed of, for example, a servo motor. For example, a sprocket is attached to an output shaft of the actuator unit 52. The sprocket is engaged with, for example, a chain that is connected with a proximal end portion of the wire 25 that is opposite to the distal end portion of the wire 25. The distal end portion of the wire 25 is connected with the distal end portion 16. With such a motive power transmission mechanism, the actuator unit 52 is capable of moving the wire 25 forward and backward. By the forward and backward movement of the wire 25, the flexible tube 24 of the endoscope inserting unit 21 can be bent in a desired direction in one plane P. The structure of the motive power transmitting mechanism is an example, and motive power may be transmitted to the wire 25 from the actuator unit 52 using another motive power transmitting mechanism, such as a mechanism using a pulley and a belt. The wire 25 is an example of the linear member.

The controller 13 is formed of, for example, an ordinary computer, and software installed in the computer and performs various types of control on the inserting device 12. The controller 13 calculates the indicating direction of the pointer 37 from the rotation amount (rotation angle) of the dial portion 35 (rotation axis 53) sensed by the sensing unit 51. The indicating direction of the pointer 37 is indicated with, for example, the rotation angle from a reference point. In the present modification, the reference point is a position (position corresponding to the initial state) in which the pointer 37 is disposed in FIG. 5. The controller 13 sets the direction indicated by the pointer 37 as a target value, when the dial portion 35 is rotated. The controller 13 controls the actuator unit 52 such that the extending direction D1 of the distal end portion 16 of the endoscope inserting unit 21 agrees with the target value. The relation between the rotation angle of the actuator unit 52 and the bending angle of the flexible tube 24 of the endoscope inserting unit 21 is measured in advance, and the controller 13 controls the rotation angle of the actuator unit 52 such that the extending direction D1 of the distal end portion 16 of the endoscope inserting unit 21 agrees with the target value (the indicating direction of the pointer 37).

The following is the operation of the endoscope system 11 according to the present embodiment, with reference to FIG. 5.

In a medical examination, the doctor who is the user can insert the endoscope inserting unit 21 into the cavity (into the lumen) of the medical examinee (subject), to observe the inside of the cavity. In this state, as illustrated in FIG. 5, the extending direction D1 of the distal end portion 16 of the endoscope inserting unit 21 in the initial state agrees with the indicating direction of the pointer 37. The user can rotate the dial portion 35 to bend the flexible tube 24 of the endoscope inserting unit 21, and change the extending direction D1 of the distal end portion 16 to a desired direction in one plane P. For example, suppose that the endoscope inserting unit 21 is bent to a certain bent state (for example, the state illustrated in FIG. 4) from the initial state illustrated in FIG. 5. In this state, the controller 13 performs feedback control on the actuator unit 52, to cause the extending direction D1 of the distal end portion 16 of the endoscope inserting unit 21 to agree with the target value being the indicating direction of the pointer 37. With this structure, also in the present modification, the extending direction D1 of the distal end portion 16 of the endoscope inserting unit 21 agrees with the indicating direction of the pointer 37 of the bending operation unit 34, even after the endoscope inserting unit 21 is bent, in the same manner as the embodiment described above.

Accordingly, even in the state where the endoscope inserting unit 21 is inserted into the cavity of the medical examinee, the doctor who is the user is enabled to recognize the extending direction of the distal end portion 16 by checking the indicating direction of the pointer 37. In addition, because the user is enabled to recognize the indicating direction of the pointer 37 through tactile sensation at the time when his or her hand contacts the pointer 37, the user is enabled to recognize the direction observed with the distal end portion 16 without taking his or her eyes off the display unit 14.

According to the present modification, the bending operation unit 34 includes the dial portion 35 that is rotatably provided on the holding unit 17 and provided with the pointer 37, the actuator unit 52 bending the endoscope 15 in one plane P, and the controller 13 controlling the actuator unit 52 such that the extending direction of the distal end portion 16 agrees with the indicating direction of the pointer 37 of the dial portion 35.

This structure enables agreement between the extending direction D1 of the distal end portion 16 and the indicating direction of the pointer 37, also by electrical control using the controller 13. This structure reduces the number of components used for the mechanical motive power transmitting mechanism, reduces the frequency of occurrence of a malfunction, and improves the reliability of the endoscope system 11.

In the present modification, a desirable mode is a mode of using the endoscope inserting unit 21 along the direction of the central axis C, without moving the endoscope inserting unit 21 from the initial position S1, such that the indicating direction of the pointer 37 always agrees with the extending direction D1 of the distal end portion 16. In the case of the mode of moving the endoscope inserting unit 21 from the initial position S1 along the direction of the central axis C, it is desirable to use the following second modification.

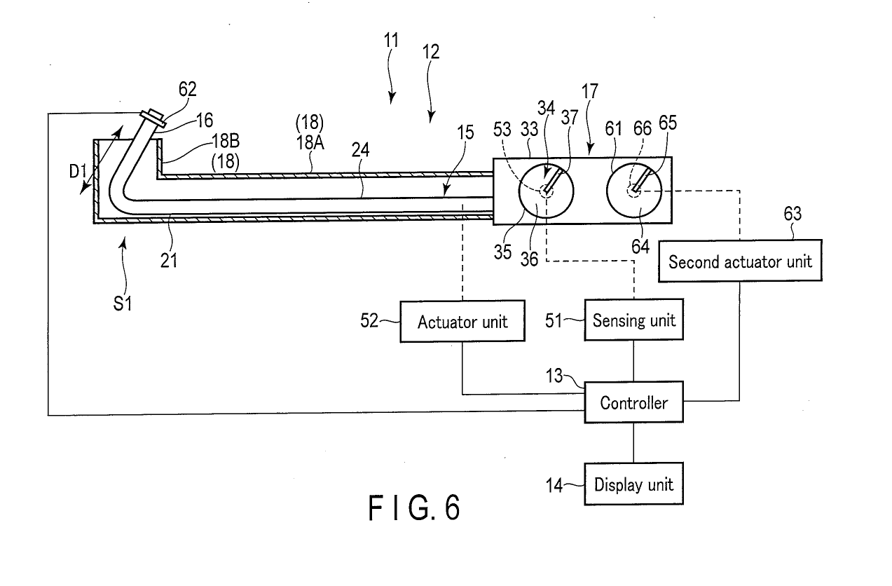

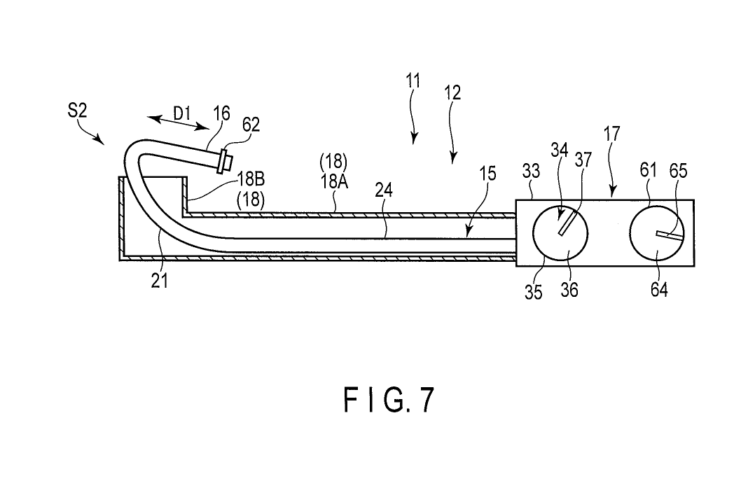

The following explains the second modification of the present embodiment, with reference to FIG. 6 and FIG. 7. The second modification explained hereinafter mainly illustrates parts that are different from the first modification, and explanation of parts common to the first and the second modifications is omitted. Although the extending direction D1 of the distal end portion 16 of the endoscope inserting unit 21 is not sensed in the second modification, the present modification has the structure in which a second sensing unit 62 is provided to always sense the extending direction D1 of the distal end portion 16 of the endoscope inserting unit 21. In the present modification, a second dial portion 61 is provided separately from the dial portion 35 to bend the endoscope inserting unit 21, to indicate the actual extending direction D1 of the distal end portion 16 of the endoscope inserting unit 21.

The bending operation unit 34 includes the dial portion 35 that is rotatable with respect to the housing 33, the sensing unit 51 sensing the rotation amount of the dial portion 35, the actuator unit 52 to move the wire 25 forward and backward to bend the endoscope inserting unit 21 in one plane P, the second dial portion 61 that is rotatable with respect to the housing 33, the second sensing portion 62 provided on the distal end portion 16 of the endoscope inserting unit 21, and the controller 13. The dial portion 35 includes the dial surface 36 that is formed flat, the pointer 37 (pointer projecting portion) provided to project from the dial surface 36, and the rotation axis 53 provided on a side opposite to the dial surface 36. The sensing unit 51 is formed of, for example, a potentiometer, and is capable of reading the rotation amount (rotation angle) of the rotation axis 53.

The second dial portion 61 is connected to the controller 13 via a second actuator unit 63 formed of a servomotor or the like. The second dial portion 61 includes a second dial surface 64 that is formed flat, a second pointer 65 (second pointer projecting portion) provided to project from the second dial surface 64, and a second rotation axis 66 provided on a side opposite to the second dial surface 64. The second pointer 65 of the second dial portion 61 is rotated by the second actuator unit 63 controlled by the controller 13, and is thereby capable of indicating the inclination (inclination with respect to the ground) of the distal end portion 16 of the endoscope inserting unit 21 in one plane P.

The second pointer 65 is useful to recognize the actual extending direction D1 of the distal end portion 16, in the case where the extending direction D1 of the distal end portion 16 is shifted from the indicating direction of the pointer 37 of the dial portion 35 when the endoscope inserting unit 21 is caused to project from the guide pipe 18 as exemplary illustrated in FIG. 7.

The second sensing unit 62 is formed of a gravity sensor (acceleration sensor). The second sensing unit 62 is capable of sensing the inclination of the distal end portion 16 of the endoscope inserting unit 21 with respect to the ground.

The controller 13 is formed of, for example, an ordinary computer, and software installed in the computer and performing various types of control on the inserting device. The controller 13 controls the bending amount of the flexible tube 24 of the endoscope inserting unit 21, by operating the actuator unit 52, based on the rotation amount (rotation angle) of the dial portion 35 (rotation axis 53) sensed by the sensing unit 51. Specifically, the controller 13 controls the actuator unit 52 such that the bending amount of the flexible tube 24 increases as the rotation amount of the dial portion 35 is increased. With this structure, when the endoscope inserting unit 21 is in the initial position S1 in the direction of the central axis C, the indicating direction of the pointer 37 agrees with the extending direction D1 of the distal end portion 16 of the endoscope inserting unit 21, in the same manner as the first modification.

By contrast, the controller 13 always senses the inclination of the distal end portion 16 of the endoscope inserting unit 21 with respect to the ground, with the electrical signal transmitted from the second sensing unit 62. The controller 13 controls the second actuator unit 63 such that the actual extending direction D1 (inclination with respect to the ground) of the distal end portion 16 of the endoscope inserting unit 21 agrees with the indicating direction of the second pointer 65 of the second dial portion 61.

The following explains the operation of the endoscope system 11 according to the present modification, with reference to FIG. 6 and FIG. 7.

In a medical consultation, the doctor who is the user can insert the endoscope inserting unit 21 into the cavity (lumen) of the medical examinee (subject), to observe the inside of the cavity. In this state, the user can operate the dial portion 35 of the bending operation unit 34, to bend the flexible tube 24 of the endoscope inserting unit 21, and change the extending direction D1 of the distal end portion 16 to a desired direction in one plane P. In this state, because the endoscope inserting unit 21 is in the initial position S1 in the direction of the central axis C, the extending direction D1 of the distal end portion 16 of the endoscope inserting unit 21 agrees with the indicating direction of the pointer 37, as illustrated in FIG. 6.

When the user operates the moving mechanism to change the position of the endoscope inserting unit 21 in the direction of the central axis C, a portion of the flexible tube 24 is bent along the guide pipe 18, even when the portion is a portion other than the bending portion that is bent by the wire 25, as illustrated in FIG. 7. When the endoscope inserting unit 21 is in a projecting position S2 in which the endoscope inserting unit 21 projects from the initial position S1 like this, the actual extending direction D1 of the distal end portion 16 of the endoscope inserting unit 21 is shifted from the indicating direction of the pointer 37 of the dial portion 35. However, in the present modification, the controller 13 is capable of controlling the second actuator unit 63 based on information from the second sensing unit 62, to indicate the actual extending direction D1 of the distal end portion 16 of the endoscope inserting unit 21, with the second pointer 65. Accordingly, the doctor who is the user is enabled to recognize the actual extending direction D1 of the distal end portion D1, by checking the indicating direction of the second pointer 65. In addition, because the user is enabled to recognize the indicating direction of the second pointer 65 with the tactile sensation at the time when his or her hand contacts the second pointer 65, the user is enabled to recognize the direction observed with the distal end portion 16 without taking his or her eyes off the display unit 14.

According to the present modification, even when the endoscope inserting unit 21 is in the projecting position S2 in which the endoscope inserting unit 21 projects from the initial position S1 to the outside of the guide pipe 18, the user is enabled to recognize the actual extending direction D1 of the distal end portion 16. This structure enables the user to intuitively recognize the direction observed with the distal end portion 16. This structure prevents the user from confusing the orientation during observation of the inside of the cavity of the subject, and improves user convenience.

The embodiment and the modifications have been specifically explained above with reference to drawings, but the present invention is not limited to the embodiment described above. The present invention can be realized with modified constituent elements within a range not departing from the gist thereof. Examples of the cavity of the medical examinee (subject) include a paranasal sinus, but the endoscope system 11 may be used for observation of other cavities such as the urethra and the urinary bladder, as a matter of course. Specifically, even when no (or few) landmarks exist, such that the case where the observation target of the subject is the urethra or the urinary bladder, the user is enabled to intuitively recognize the direction of the endoscope by tactile sensation via the pointer 37. This structure enables the user to easily perform examination and screening on specific regions. In the embodiment and modifications described above, the dial portion 35 (pointer 37) and the second dial portion 61 (second pointer 65) are provided to correspond to bending in one plane P, but additional dial portions and pointers may be provided to correspond to bending in another plane (for example, a plane orthogonal to the plane P). In addition, constituent elements in the different embodiments described above may be properly used in combination to achieve an endoscope system 11.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope system comprising:
a holding unit;
a guide pipe extending from the holding unit;
an endoscope including a distal end portion capable of acquiring an image, the endoscope being inserted through an inside of the guide pipe and bendable in at least one plane;
a bending operation unit rotatably provided on the holding unit and capable of adjusting a bending angle of the endoscope; and
a pointer provided on the bending operation unit and indicating an extending direction of the distal end portion in the plane.

2. The endoscope system according to claim 1, wherein an indicating direction of the pointer is recognizable by a user by tactile sensation.

3. The endoscope system according to claim 2, wherein
the endoscope includes a linear member to adjust the bending angle, and
the bending operation unit includes:
a dial portion rotatably provided on the holding unit and provided with the pointer; and
a converting portion to convert rotation of the dial portion into forward and backward movement of the linear member.

4. The endoscope system according to claim 2, wherein the bending operation unit includes:
a dial portion rotatably provided on the holding unit and provided with the pointer;
an actuator unit bending the endoscope in the at least one plane;
a sensing unit sensing a rotation amount of the dial portion; and
a controller controlling the actuator unit such that the extending direction of the distal end portion agrees with the indicating direction of the pointer of the dial portion, based on the rotation amount of the dial portion sensed by the sensing unit.

5. The endoscope system according to claim 1, further comprising a display unit capable of displaying the image acquired by the distal end portion.
